(19) United States
(12) Patent Application Publication
(10) Pub. No.: US 2003/0157534 A1
(43) Pub. Date: Aug. 21, 2003

(54) DNA immunization with libraries of minigenes encoding degenerate variants of major histocompatability class l restricted epitopes

(76) Inventors: Manuel Engelhorn, New York, NY (US); Alan N. Houghton, New York, NY (US); Jose Alejandro Guevara, New York, NY (US)

Correspondence Address: OPPEDAHL AND LARSON LLP, P O BOX 5068, DILLON, CO, 80435-5068, US

(21) Appl. No.: 10/331,163

(22) Filed: Dec. 26, 2002

Related U.S. Application Data
(60) Provisional application No. 60/343,735, filed on Dec. 26, 2001.

(51) Int. Cl. C12Q 1/68; C07H 21/04

(52) U.S. Cl. 435/6; 536/23.1

(57) ABSTRACT

Therapeutic libraries of minigenes encoding at least one characteristic epitope of a target antigen and degenerate variants of major histocompatability class I restricted epitopes are prepared by constructing a population of minigenes of comparable structure (that is having parallel components at each location in the minigene), each species in the population encoding at least one characteristic epitope of the target antigen and one of a plurality a degenerate variants of an MHC Class I-restricted epitope; screening the population of minigenes in an in vitro or in vivo system to confirm the induction of immunological response against the target antigen; and optionally repeating a screening step on one or more subpopulations of the minigenes to select for immunologically-effective subpopulations, which may comprise as few as a single species of the minigene construct.

This application claims the benefit of U.S. Provisional Application No. 60/343,735, filed Dec. 26, 2001, which is incorporated herein by reference.

### BACKGROUND OF THE INVENTION

This application relates to a method for DNA immunization using libraries of minigenes encoding degenerate variants of major histocompatability class-I restricted epitopes, thus providing a novel approach to development of vaccines with increased potency. This approach may be used for vaccines against cancer, where antigens are weak, but also against infectious agents including bacteria, viruses and parasites.

Previously described approaches for immunizing against defined epitopes include immunization with a synthetic peptide or a minigene DNA construct encoding the epitope. When dealing with weak epitopes, ways to design more potent vaccine design must be found. Furthermore, in the case of self-derived epitopes neither of these strategies specifically addresses the likeliness of immunological anergy or tolerance. A further development, heteroclitic immunization, makes use of structural information about the MHC-peptide interface to develop improved epitopes which bind MHC with higher affinity than the endogenous counterpart. Immunization with heteroclitic epitopes often yields stronger priming of T cell responses to the endogenous peptide.

Another site of possible epitope improvement is the peptide-TCR interface. Unlike the MHC-epitope contacts, there are no rules as to i) which amino acid positions of the peptide are actually involved, and ii) which side chains are preferred at the involved positions. Thus, rational design of epitopes improved for TCR contacts is not feasible. The present invention circumvents this limitation.

### SUMMARY OF THE INVENTION

The present invention provides a method for preparing a therapeutic library of minigenes encoding at least one characteristic epitope of a target antigen and degenerate variants of major histocompatability class I restricted epitopes comprising the steps of:

(a) constructing a population of minigenes of comparable structure (that is having parallel components at each location in the minigene), each species in the population encoding at least one characteristic epitope of the target antigen and one of a plurality a degenerate variants of an MHC Class I-restricted epitope;

(b) screening the population of minigenes in an in vitro or in vivo system to confirm the induction of immunological response against the target antigen; and

(c) if step (b) is positive, optionally repeating a screening step on one or more subpopulations of the minigenes to select for immunologically-effective subpopulations, which may comprise as few as a single species of the minigene construct.

The present invention further provides immunologically-effective minigene libraries each comprising a population of minigenes of comparable structure (that is having parallel components at each location in the minigene), each encoding at least one characteristic epitope of the target antigen and one of a plurality a degenerate variants of an MHC Class I-restricted epitope.

The present invention further provides a method for inducing an immune response to a target antigen comprising the steps of administering a DNA vaccine comprising at least one species of minigene encoding at least one characteristic epitope of the target antigen and a degenerate variant of an MHC Call I-restricted epitope. This method may be practiced using a library of minigenes as the DNA vaccine, in which each species of minigenes encodes the characteristic epitope of the target antigen and one of a plurality of degenerate variants of an MHC Call I-restricted epitope.

### BRIEF DESCRIPTION OF THE DRAWINGS

FIG. 1 shows the amino acid sequence encoded by a minigene used to exemplify the invention.

FIG. 2 shows a schematic of the method of the invention.

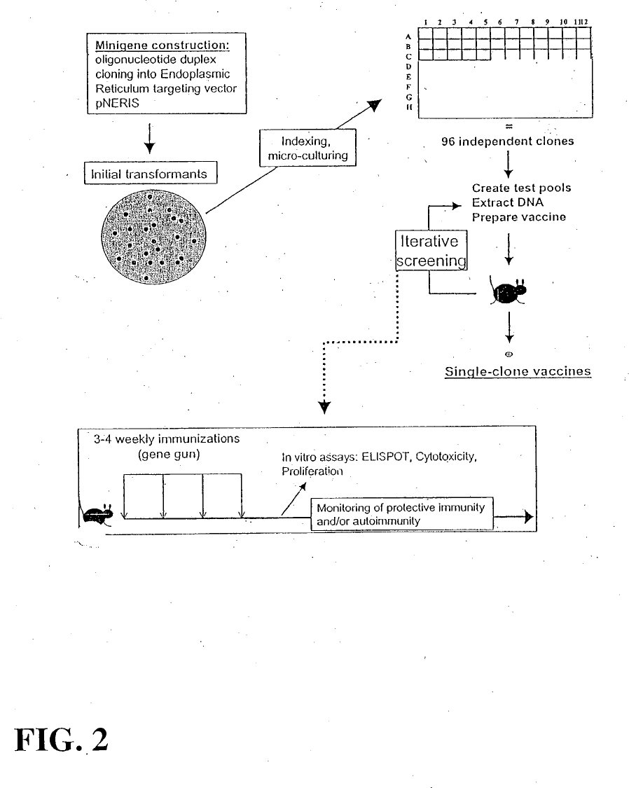

### DETAILED DESCRIPTION OF THE INVENTION

The present invention relates to a method for inducing an immune response to a target antigen by DNA immunization with a minigene or minigene library which encodes at least one characteristic epitope of the target antigen and at least one variant of a major histocompatability class-I restricted epitope. In one embodiment of the invention, a single species found by iterative screening to be effective to produce an enhanced immune response is utilized. In another embodiment, a mixture (or library) containing multiple variants of the MHC Class-I restricted epitope is used for immunization.

As used in the specification and claims of this application, the tern “minigene” refers to a heterologous gene construct wherein one or more nonessential segments of a gene are deleted with respect to the naturally-occurring gene under the control of a eukaryotic promoter. Typically, deleted segments are intronic sequences of at least about 100 basepairs to several kilobases, and may span up to several tens of kilobases or more. Isolation and manipulation of large (i.e., greater than about 50 kilobases) targeting constructs is frequently difficult and may reduce the efficiency of transferring the targeting construct into a host cell. Thus, it is frequently desirable to reduce the size of a targeting construct by deleting one or more nonessential portions of the gene. Typically, intronic sequences that do not encompass essential regulatory elements may be deleted. Frequently, if convenient restriction sites bound a nonessential intronic sequence of a cloned gene sequence, a deletion of the intronic sequence may be produced by: (1) digesting the cloned DNA with the appropriate restriction enzymes, (2) separating the restriction fragments (e.g., by electrophoresis), (3) isolating the restriction fragments encompassing the essential exons and regulatory elements, and (4) ligating the isolated restriction fragments to form a minigene wherein the exons are in the same linear order as is present in the germline copy of the naturally-occurring gene. Alternate methods for producing a minigene will be apparent to those of skill in the art (e.g., ligation of partial genomic clones which encompass essential exons but which lack portions of intronic sequence). Most typically, the gene segments comprising a minigene will be arranged in the same linear order as is present in the germline gene, however, this will not always be the case. Some desired regulatory elements (e.g., enhancers, silencers) may be relatively position-insensitive, so that the regulatory element will function correctly even if positioned differently in a minigene than in the corresponding germline gene. For example, an enhancer may be located at a different distance from a promoter, in a different orientation, and/or in a different linear order. For example, an enhancer that is located 3′ to a promoter in germline configuration might be located 5′ to the promoter in a minigene. Similarly, some genes may have exons which are alternatively spliced at the RNA level, and thus a minigene may have fewer exons and/or exons in a different linear order than the corresponding germline gene and still encode a functional gene product. A cDNA encoding a gene product may also be used to construct a minigene. However, since it is often desirable that the heterologous minigene be expressed similarly to the cognate naturally-occurring nonhuman gene, transcription of a cDNA minigene typically is driven by a linked gene promoter and enhancer from the naturally-occurring gene.

The target antigen against which the invention induces an immune response may be any antigen for which a therapeutic benefit is derived as a result of the induction of an immune response, including antigens associated with pathogenic microorganisms and antigens associated with cancers. The invention is particularly applicable for inducing an immune response to inherently non-immunogenic or poorly immunogenic antigens. Specific, non-limiting examples of target antigens include gp75/TRP-1, TRP-2, tyrosinase, gp100/pMel17 on melanoma; prostate specific membrane antigen, prostate specific antigen and prostate stem cell antigen on prostate cancers; HER2/neu and the mucin MUC1 on breast cancers; CD19 and CD20 on malignancies of B lymphocyte origin; MAGE, BAGE and GAGE, NY-ESO-1 and other “cancer-testes” antigens on a variety of cancer types; gene products from the human immunodeficiency virus-1; angiogenic factors (such as VEGF, bFGF, angiopoietins, their cognate cell surface receptors, and ELR C-X-C chemokines); tumor suppressor genes such as p53; dipeptidyl peptidase IV and fibroblast activation protein-1.

As used in the specification and claims of this application, the phrase “inducing an immune response” refers to both the stimulation of a new immune response or to the enhancement of a pre-existing immune response to a target antigen. The immune response may be a cytolytic T-cell mediated cellular immune response or a B-cell mediated humoral response, or some combination thereof.

The term “subject” refers to the living organism being treated to induce an immune response. The subject will generally be mammalian or avian. Preferred “subjects” are human patients.

In the method of the invention, an immune response to a target antigen is induced in a subject comprising administering to the subject a vaccine composition in an amount sufficient to induce an immune response to the target antigen, wherein the vaccine composition comprises either (1) DNA minigenes of a single type comprising at least one sequence encoding an epitope of the target antigen and a variant of an MHC-Class I restricted epitope or (2) a mixture (or library) DNA minigene species, each comprising at least one sequence encoding an epitope of the target antigen and a variant of an MHC-Class I restricted epitope. one suitable mode of administration is subcutaneous injection of particles coated with the nucleic acid mixture using a GENE GUN. Thus, one embodiment of the vaccine composition comprises carrier particles coated with the pool of nucleic acid, i.e. with a mixture comprising a plurality of nucleic acid species encoding a plurality of mutant forms of the target antigen. The expressed mutant proteins or peptides are immunogenic and stimulate an immune response to the target antigen, even in the case where the target antigen is inherently non-immunogenic or only weakly immunogenic in the subject. The carrier particles used in this composition may be any of various types of particles known for use in this purpose, including without limitation gold, clay and tungsten. The particles suitably are from 0.5 to 2 microns in diameter to facilitate transdermal injection.

Other delivery systems which can be used to administer the nucleic acid vaccine compositions of the invention include the pressure delivery systems, for instance the BIOJECT system which delivers vaccines using carbon dioxide pressure cartridges. In this case, particles are not required, but can be used. The vaccine compositions can also be administered without a particle carrier using non-pressurized systems, for example syringe needles. Administration could also be accomplished using a mucosal route (e.g, a nasal spray). The pool of mutated DNA may also be incorporated into a viral vector, which is then associated with particles for administration by the routes described above.

The vaccine composition above may be administered in a liquid carrier by subcutaneous injection. For use in a Gene Gun, however, the composition is suitably packaged into therapeutic administration units, sometimes referred to as “bullets”. This is accomplished by drawing the composition into the lumen, a thin hollow tube, and then cutting the tube into lengths containing about 1 μg of nucleic acid.

In the example of vaccination against melanoma, melanosomal glycoproteins are suitable target antigens because their expression is restricted to the tumor and healthy melanocytes, a nonessential tissue. A K^{b}-restricted nonapeptide from the mouse melanosomal glycoprotein TYRP-2, SVYDFFVWL (positions 180-188), has been reported to be efficiently presented by melanomas. Immunity to this epitope in the mouse model is desirable in the perspective of melanoma therapy. In order to obtain an epitope with improved TCR contacts, we created an indexed library of minigene variants where amino acid positions other than the major anchors to MHC were randomized. Anchor residues in nonapeptides binding to K^{b} are at residues 6 and 9. We randomized position 8, a putative TCR contact residue. Mice immunized with the entire library of random variants displayed autoimmune coat hypopigmentation, a sign that significant immune responses were achieved.

The strategy described here represents an approach to design a vaccine through in vitro or in vivo selection using minigene libraries. The optimal vaccine is identified by iterations with smaller and smaller library subsets. This approach can be used to identify peptide or minigene vaccines against antigens, particularly weak antigens, expressed by cancers and infectious pathogens. It also has the potential to improve immunization against carcinogenic fusion gene products resulting from chromosomal rearrangements in cancers such as sarcoma and leukemias.

### EXAMPLE 1

Minigene Constructs and Indexed Library

Minigenes were constructed in the pNERIS parental vector plasmid. This vector encodes the first 13 amino acids of the endoplasmic reticulum insertion sequence (ERIS) from the adenoviral protein E3. Ligation of appropriately sticky-ended oligonucleotide duplexes between the unique PstI and XhoI sites of this vector yielded final constructs expressing the following polypeptide sequences (ERIS underlined; epitope italicized): MRYMILGLLALAAVCSASVYDFFVXL.

Position 8 of the epitope is randomized. A separate construct was made where X is tryptophane, which corresponds to the wild-type sequence. Wild type epitope-encoding oligonucleotide sequences were: 5′-GTGTGCAGCGCCAGCGTGTACGACTTCTTCGTGTGGCTGTGAC-3′ (top strand); and 5′-TCGAGTCACAGCCATACGAAGAAGTCGTACACGCTGGCGCTGCACACTGCA-3′ (bottom strand).

Several clones with the confirmed correct DNA insert were obtained. One was arbitrarily chosen for further work. Randomized epitope-encoding oligonucleotide sequences were: 5′-GTGTGCAGCGCCAGCGTGTACGACTTCTTCGTGNNSCTGTGAC-3′ (top strand; S═C or G); and 5′-TCGAGTCACAGSNNTACGAAGAAGTCGTACACGCTGGCGCTGCACACTGCA-3′ (bottom strand).

Several clones were picked at random and sequenced to confirm divergence at position 8.

96 clones obtained from the randomizing ligation reaction were picked and individually microcultured as an indexed library. The pool of all 96 clones was also grown as bulk and its plasmid DNA was prepared for immunization.

Random variations can be introduced at other positions, for example position 1 or 7 using the same basic technique.

### EXAMPLE 2

Preparation of DNA for Genetic Immunization

DNA from the plasmid pools was coated onto 0.8-1.5 mm gold particles (Alfa Aesar) at a ratio of 100 mg DNA for 50 mg gold. The gold/DNA precipitate was deposited in teflon tubing which was cut in “bullets” each representing 1 mg of DNA. Bullets were loaded in a helium-pressure PowderJect gene gun for genetic immunization of mice by delivery of the DNA-coated gold to the epidermis.

### EXAMPLE 3

Immunization Protocol

C57BL/6 mice were depilated and immunized four times at weekly intervals receiving each time 4 mg of DNA, delivered to 4 sites on the abdomen. There were 10 mice per group. Control animals received the unmutated murine minigene.

### EXAMPLE 4

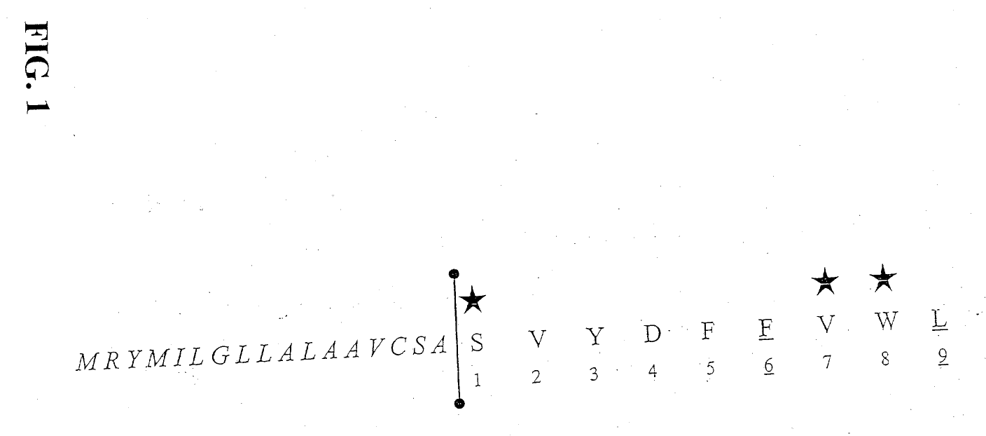

Genetic immunization with a pool of variants of the SVYDFFVWL TYRP-2 epitope was shown to induce autoimmunity. An indexed library of randomized minigenes was constructed. It consisted of 96 discrete clones where the codon for position 8 of the TYRP-2 ₁₈₀₋₁₈₈ nonapeptide was left degenerate by oligonucleotide design. FIG. 1 shows the amino acid sequence encoded by the minigenes of this example. The adenovirus E3 protein endoplasmic reticulum insertion sequence (ERIS) is shown in italics. The numbers refer to epitope residues after release from the ERIS. Major anchors to the murine K^{b} major histocompatability class I molecule are underlined. Stars indicate the positions (1, 7 and 8) for which randomized minigene libraries are preferably generated.

The size of the library ensures representation of codons for all 20 amino acids at position 8. We immunized C57BL/6 mice with the entire pooled library or the control minigene encoding the wild-type peptide. We monitored autoimmune coat hypopigmentation as a readout for effective immunity. Hypopigmentation was observed only in animals immunized with the pooled library.

Individual clones with high potency are selected from the therapeutic library through iterative screening. One exemplary protocol for such screening is illustrated in FIG. 3. Minigenes encoding the randomized epitope of interest are generated with an endoplasmic reticulum targeting vector. Following microculturing of all clones to generate an indexed library, animals are immunized with the whole pooled library or iteratively with smaller and smaller subsets of it, following an appropriate immunization schedule. Single-clone vaccines with enhanced potency can be identified by multiple rounds of screening.

1. A method for preparing a therapeutic library of minigenes encoding at least one characteristic epitope of a target antigen and degenerate variants of major histocompatability class I restricted epitopes comprising the steps of:
(a) constructing a population of minigene species of comparable structure, each species encoding at least one characteristic epitope of the target antigen and one of a plurality a degenerate variants of an MHC Class I-restricted epitope; and
(b) screening the population of minigenes in an in vitro or in vivo system to confirm the induction of immunological response against the target antigen.

2. The method of claim 1, wherein if step (b) is positive, the method further comprises the step of repeating the screening step on one or more subpopulations of the minigenes to select for a library comprising an immunologically-effective subpopulation.

3. The method of claim 1, wherein the target antigen is selected from the group consisting of gp75/TRP-1, TRP-2, tyrosinase, gp100/pMel17, prostate specific membrane antigen, prostate specific antigen and prostate stem cell antigen, HER2/neu, the mucin MUC1, CD19, CD20, MAGE, BAGE, GAGE, NY-ESO-1, gene products from the human immunodeficiency virus-1, angiogenic factors, tumor suppressor genes, dipeptidyl peptidase IV and fibroblast activation protein-1.

4. A minigene library comprising in a mixture a plurality of species of minigenes of comparable structure, each species of minigene encoding a characteristic epitope of a selected target antigen and an MHC-Class I restricted epitope, wherein the plurality of species taken together encode a plurality of degenerate MHC-Class I restricted epitopes, wherein the minigene library is effective to induce an immune response to the selected target antigen when administered to a subject in which it is expressed.

5. The minigene library of claim 4, wherein the target antigen is selected from the group consisting of gp75/TRP-1, TRP-2, tyrosinase, gp100/pMel17, prostate specific membrane antigen, prostate specific antigen and prostate stem cell antigen, HER2/neu, the mucin MUC1, CD19, CD20, MAGE, BAGE, GAGE, NY-ESO-1, gene products from the human immunodeficiency virus-1, angiogenic factors, tumor suppressor genes, dipeptidyl peptidase IV and fibroblast activation protein-1.

6. A method of inducing an immune response in an individual suffering from a health condition characterized by the presence of a target antigen, comprising the step of administering to the individual a minigene library comprising in a mixture a plurality of species of minigenes of comparable structure, each species of minigene encoding a characteristic epitope of the target antigen and an MHC-Class I restricted epitope, wherein the plurality of species taken together encode a plurality of degenerate MHC-Class I restricted epitopes, in an amount effective to induce an immune response to the target antigen when administered to a subject and expressed.

7. The method of claim 6, wherein the target antigen is selected from the group consisting of gp75/TRP-1, TRP-2, tyrosinase, gp100/pMel17, prostate specific membrane antigen, prostate specific antigen and prostate stem cell antigen, HER2/neu, the mucin MUC1, CD19, CD20, MAGE, BAGE, GAGE, NY-ESO-1, gene products from the human immunodeficiency virus-1, angiogenic factors, tumor suppressor genes, dipeptidyl peptidase IV and fibroblast activation protein-1.
